(19) United States
(12) Patent Application Publication
(10) Pub. No.: US 2025/0180574 A1
(43) Pub. Date: Jun. 5, 2025

(54) GRAPHENE SUPPORTED CRYO-ELECTRON MICROSCOPY GRID

(71) Applicant: The Regents of the University of Michigan, Ann Arbor, MI (US)

(72) Inventors: Uhn-Soo Cho, Ann Arbor, MI (US); Eungjin Ahn, Ann Arbor, MI (US)

(21) Appl. No.: 18/833,720

(22) PCT Filed: Feb. 1, 2023
(86) PCT No.: PCT/US23/61737

§ 371 Date: Jul. 26, 2024

Related U.S. Application Data
(60) Provisional application No. 63/305,411, filed on Feb. 1, 2022.

(51) Int. Cl. G01N 33/68; H01J 37/26

(52) U.S. Cl. CPC G01N 33/6803; H01J 37/26

(57) ABSTRACT

The present disclosure provides to electron microscopy grids and methods, kits, and systems for using and fabricating thereof. More particularly, the present disclosure provides electron microscopy grids comprising a single first layer comprising graphene, wherein the graphene covers greater than 90% of the grid, and a second layer comprising a binding agent configured to interact with a target protein.

### CROSS-REFERENCE TO RELATED APPLICATIONS

This application claims the benefit of U.S. Provisional Application No. 63/305,411, filed Feb. 1, 2022, the content of which is herein incorporated by reference in its entirety.

### FIELD

The present invention relates to electron microscopy grids and methods, kits, and systems for using and fabrication thereof.

### BACKGROUND

Cryo-electron microscopy (cryo-EM) can provide 3D structural information of biological molecules and assemblies by imaging non-crystalline specimens (single particles), albeit at substantially lower resolutions than crystallography. Though the overall strategy has not changed significantly over the years, recent technological advances in sample preparation, computation, and instrumentation facilitates use of single-particle cryo-EM for solving near-atomic-resolution macromolecular structures. One existing barrier to structure determination of macromolecular complexes is protein complex purification and isolation as protein complexes are difficult to express recombinantly and to purify in large enough quantities for cryo-EM study. In addition, several cryo-EM structures determined following in vitro reconstitution did not agree with known biological functions of these protein complexes raising doubt that in vitro reconstituted protein complexes represent the physiologically relevant conformation.

### SUMMARY

Provided herein are grids for cryo-electron microscopy (EM) comprising a single first layer comprising graphene and a second layer comprising a binding agent configured to interact with a target protein, wherein the graphene covers greater than 90% (e.g., greater than about 91%, greater than about 92%, greater than about 93%, greater than about 94%, greater than about 95%, greater than about 96%, greater than about 97%, greater than about 98%, or greater than about 99%) of the grid.

Also provided are methods for preparing a target protein for structure analysis comprising: incubating the disclosed grids with a sample comprising the target protein; removing excess sample; and visualizing the target protein on the grid by electron microscopy. In some embodiments, the methods further comprise vitrifying the target protein deposited on the grid. In some embodiments, the sample is partially purified prior to the incubation.

Further provided are methods for manufacturing the disclosed grids comprising: transferring a graphene sheet in aqueous solution to the surface of a grid substrate to form a graphene coated grid, wherein the grid substrate comprises holey patterned amorphous carbon, amorphous carbon coated copper, amorphous carbon coated gold, gold, or silicon nitride; removing the aqueous solution; drying the graphene coated grid; oxidizing the graphene coated grid; and crosslinking the binding agent to the oxidized graphene grid. In some embodiments, the graphene sheet comprises poly (methyl methacrylate) (PMMA) polymer and the method further comprises removal of the PMMA. In some embodiments, the removal of the PMMA comprises washing with a solvent, heating with or without vacuum, or a combination thereof.

In some embodiments, the binding agent comprises a polypeptide. In some embodiments, the binding agent comprises calmodulin. In some embodiments, the binding agent comprises an antibody, a nanobody, or a fragment, derivative, or analog thereof.

In some embodiments, the binding agent is attached to the first layer by a linker. Thus, in some embodiments, the binding agent may further comprise a linker. In some embodiments, the linker is a flexible linker. In some embodiments, the linker comprises a glycine-serine rich polypeptide. In some embodiments, the linker comprises polyethylene glycol.

In some embodiments, the linker further comprises a crosslinking site. In some embodiments, the crosslinking site comprises more than one lysine residues or one or more amino groups.

In some embodiments, the target protein comprises an affinity tag configured to interact with the binding agent. In some embodiments, the affinity tag comprises a CBP-tag or an ALFA-tag.

In some embodiments, the sample comprises a biological sample and the target protein is obtained from the biological sample (e.g., a cell lysate). In some embodiments, the sample is from a subject and the target protein is obtained the subject.

In some embodiments, the grid comprises holey patterned amorphous carbon, amorphous carbon coated copper, amorphous carbon coated gold, gold, or silicon nitride as substrate for the first layer.

Other aspects and embodiments of the disclosure will be apparent in light of the following detailed description.

### BRIEF DESCRIPTION OF THE DRAWINGS

FIG. 1 is a schematic of an exemplary plasma jet system (left) with photographs of a plasma circuit (top right) and a plasma jet system (bottom right).

FIG. 2 is a schematic and images of steps for making a graphene grid. Green dash square in photographs denote the PMMA/graphene region.

FIG. 3 is a schematic of the step-wise production scheme of the Graffendor grid (top), genetic modification of calmodulin for use in the exemplary Graffendor grid (middle), and endogenous sample preparation for the Graffendor grid (bottom).

FIGS. 4A-4H are top-side images (FIGS. 4A-4D) from PMMA/graphene-coated Quantifoil grids and bottom-side images (FIGS. 4E-4H) from PMMA-free graphene coated Quantifoil grids after rinsing/baking steps. FIGS. 4A and 4B are SEM images of PMMA/graphene-coated EM grid in different magnifications. FIG. 4C is a BF TEM image and FIG. 4D is the corresponding electron diffraction pattern of the PMMA/graphene-coated Quantifoil grid. FIG. 4E is an SEM image of PMMA-free graphene grids in different magnifications. FIG. 4G is a BF TEM image and FIG. 4H is the corresponding electron diffraction pattern of the PMMA-free graphene grid.

FIG. 5A is Raman spectra of commercial graphene monolayer (left), commercial Quantifoil grid (center), and graphene-coated grid (right). FIG. 5B is AFM characterization of the graphene-coated Quantifoil grid with 3 different imaging modes; height (left), DMT modulus (center), and adhesion (right).

FIG. 6A is purified protein complex from the yeast using the TAP-tag purification system (the APC/C complex and the SWI/SNF complex) as test specimens. SDS-PAGE gels were stained both Coomassie and silver staining. 10-fold diluted sample from the IgG elution was applied to the Graffendor grid. FIG. 6B is a representative cryo-EM micrographic image of the APC/C complex. White circles indicate particles of the APC/C complex. FIG. 6C is the 2D classification of the APC/C complex (total ˜9K particles). FIG. 6D is the ab-initio 3D reconstruction image of the APC/C complex.

FIG. 7 is the TAP-tag with the ALFA-tag (C-terminal or N-terminal tagging) on the target protein. NbALFA may be genetically modified the same strategy with the calmodulin.

### DETAILED DESCRIPTION

Sample and grid preparation present potential bottlenecks in protein structure determination using cryo-EM. Successful microscopic data acquisition requires the optimization of specimen preparation procedures, such as selecting grid types/treatment, and finding the best blotting conditions. Sample preparation, particularly for the macromolecular multi-protein complexes, requires expression and purification of the target proteins in the recombinant systems, such as E. coli, insect cells, or mammalian cell expression system. Three challenges are encountered when preparing macromolecular multi-protein complexes samples from a recombinant system. First, all complex components must be expressed and purified. Missing one or two subunits fail to assemble the intact holo-complex, or, sometimes, proteins are misfolded, non-functional, or degraded by the host cells. Second, isolating multi-protein assembly requires multiple purification steps, which not only demand time and effort, but also drop the yield dramatically below the amount needed for the structural study or fail to produce a quantity of each component for assembly at the correct stoichiometry. Third, in many cases, the multi-component protein complexes are highly dynamic and often driven by weak interactions, such that known methods fail to capture all components within the holo-complex. Therefore, the structures determined by the recombinant system may not represent the physiologically relevant and biologically active form of the complex.

Disclosed herein is a graphene-based affinity cryo-EM grid for the endogenous protein complexes (the Graffendor grid; GFD). The graphene-based affinity grids comprise a single layer of graphene with full-coverage or nearly full-coverage (e.g., greater than 90%) across the entire surface of the grid. In addition, a binding agent (e.g., an antibody) is directly linked to the grid resulting in attraction of target protein complexes on the grid surface, prevention of protein loss during grid preparation, prevention of protein denaturation, minimization of particle movement during image collection and better density control of protein complexes on the grid.

The disclosed grids, methods, and systems overcome the challenge of low copy number of the endogenous proteins, and also other hurdles in cryo-EM sample preparation, such as ice thickness, beam-induced particle motion during image collection, preferred particle distribution and orientation, and protein denaturation due to the exposure in the air-water interface. Since the target protein of interest is immobilized by the binding agents on the graphene-coated grids, (a) longer blotting does not make an empty hole, (b) beam-induced particle movement is reduced by the immobilization, (c) preferred particle distribution and orientation are less likely due to the evenly coated binding agents on the graphene grid and a long flexible linker between the binding agents and graphene surface (˜100 Å distance), and (d) particles are not exposed to the air-water interface due to the immobilization on the graphene grid.

Graffendor grids can utilize very small amounts of a recombinant target protein to determine the protein structure and are also capable of utilizing endogenous proteins which maintain the physiologically active form. More significantly, protein complexes unable to be expressed and/or purified using the traditional recombinant systems may be available for structural study using the Graffendor grid. The Graffendor grid can be used to monitor and observe patient-oriented protein structures by directly isolating protein of interest from human patient cells, tissues, organs or other samples, facilitating comparison of target protein structures from healthy individuals, and patients having or suspected of having a disease or disorder which alters the target protein structure or function.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “amino,” as used herein, refers to an —NH₂ group, a —NHR group, or an —NR₂ group, wherein R is an alkyl group. “Alkyl,” as used herein, means a straight or branched, saturated hydrocarbon chain.

The term “antibody,” as used herein, refers to a protein that is endogenously used by the immune system to identify and neutralize foreign objects, such as bacteria and viruses. Typically, an antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2, and CH3) regions, and each light chain contains one N-terminal variable (VL) region and one C-terminal constant (CL) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other. The variable regions of each pair of light and heavy chains form the antigen binding site of an antibody. The VH and VL regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FRI, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)). The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains, (ii) a F(ab′)2 fragment, which is a bivalent fragment comprising two Fab fragments linked by a disulfide bridge at the hinge region, (iii) a Fv fragment consisting of the VL and VH domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a domain antibody (dAb), which is an antibody single variable region domain (VH or VL) polypeptide that specifically binds antigen.

A “nanobody,” as used herein, refers to polypeptides comprising the variable region of a heavy chain of an antibody. A nanobody is functionally the same as a single domain antibody consisting of only one heavy chain variable region. It is the smallest antigen-binding fragment with complete function. In general, the antigen-binding properties of a nanobody can be described by three variable regions (CDRs) divided by four framework regions (FRs) with the general structure as shown below:

FR1-CDR1-FR2-CDR2-FR3-CDR3-FR4

in which FRI to FR4 refer to framework regions 1 to 4, respectively, and in which CDR1 to CDR3 refer to the complementarity determining regions 1 to 3. The amino acid sequences of four FRs are relatively conservative and do not directly participate in binding reactions. The CDRs normally form a loop structure in which the B-sheets formed by the FRs therebetween are spatially close to each other, constituting the antigen-binding site of the nanobody. The amino acid sequences of the same type of nanobodies can be compared to determine which amino acids constitute the FR or CDR regions. The present invention includes not only intact nanobodies but also fragment(s) of immunologically active nanobody or fusion protein(s) formed from nanobodies with other sequences. Therefore, the present invention also includes fragments, derivatives, and analogs of the nanobodies.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies.

As used herein, the term “sample” is used in its broadest sense. In one sense, it is meant to include a specimen obtained from any source, including biological and environmental samples. Biological samples may be obtained from animals (including humans) and encompass fluids, solids, tissues, and gases. Such examples are not however to be construed as limiting the sample types. Preferably, a sample is a fluid sample such as a liquid sample. Examples of liquid samples that may be assayed include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. Samples can include a combination of liquids, solids, gasses, or any combination thereof (e.g., a suspension of lysed or unlysed cells in a buffer or solution). Samples can comprise biological materials, such as cells, microbes, organelles, and biochemical complexes. Liquid samples can be made from solid, semisolid, or highly viscous materials, such as soils, fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation.

A “subject” may be human or non-human and may include either adults or juveniles (e.g., children). Moreover, subject may mean any living organism, preferably a mammal (e.g., human or non-human). Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### EM Grid

The present disclosure provides grids for cryo-electron microscopy (cryo-EM). The grids comprise a single first layer comprising graphene and a second layer comprising a binding agent configured to interact with a target component (e.g., target protein). The graphene first layer may provide high coverage of graphene (e.g., greater than 90% or greater than 95%), particularly in comparison to grids that deposit a layer of graphene oxide.

In some embodiments, the target component may be any component of a cell or derived from a cell or fragments of a cell. The target component may comprise any biomolecule or structure from or derived from a cell, including, but not limited to, macromolecules such as proteins and nucleic acids, biomolecular complexes such as a ribosome, structures such as membranes and organelles, and extracellular components, such as extracellular vesicles. In select embodiments, the target component is a multi-protein complex.

“Binding agent” is used herein to refer to a species (e.g., protein, nucleic acid, carbohydrate) that binds to and forms a complex with the target component (e.g., target protein). The binding agent specifically binds the target component. Binding agents include antibodies, as well as antigen-binding fragments thereof and other various forms and derivatives thereof as are known in the art, and other molecules comprising one or more antigen-binding domains that bind to an antigen molecule or a particular site (epitope) on the antigen molecule. In addition to antigen and antibody specific binding pairs, other specific binding agents can include biotin and avidin (or streptavidin), carbohydrates and lectins, complementary nucleotide sequences, effector and receptor molecules, cofactors and enzymes, enzymes and enzyme inhibitors, and the like. In some embodiments, the binding agent comprises a polypeptide. In some embodiments, the binding agent comprises an antibody, a nanobody, or a fragment, derivative, or analog thereof.

In some embodiments, the binding agent comprises calmodulin. In some embodiments, the grid comprises covalently cross-linked modified calmodulin on the graphene-coated cryo-EM grid to engage with target endogenous proteins comprising the genetically engineered affinity tag (e.g., a CBP or TAP-tag, e.g., at a C-terminus). The TAP-tag system, which comprises a calmodulin-binding peptide (CBP) and IgG with the Tobacco Etch Virus protease (TEV) cleavage site in between. The CBP-calmodulin interaction can attract CBP-containing target proteins on the grid and act as a second affinity purification as well as concentrating low copy number of particles on the grid. In select embodiments, the calmodulin is modified to replace endogenous surface exposed lysine residues to arginine residues to prevent the direct cross-linking of the binding agent on the graphene surface.

In some embodiments, the binding agent may be tethered to the grid by a linker. The linker may be of various lengths to provide greater physical separation and allow more spatial mobility between the binding agent and the grid. In some embodiments, the linker facilitates greater than about 10 Å (e.g., greater than about 25 Å, greater than about 50 Å, greater than about 75 Å, greater than about 100 Å, or more) of distance between the binding agent and the grid.

In some embodiments, the linker may comprise any amino acid and may be of any length. The linker may be less than about 100 (e.g., about 90, about 80, about 70, about 60, about 50, about 40, about 30, about 20, about 10, or about 5) amino acid residues.

In some embodiments, the linker is a flexible linker, such the binding agent can have orientation freedom in relationship to the grid. For example, a flexible linker may include amino acids having relatively small side chains, and which may be hydrophilic. Without limitation, the flexible linker may contain a stretch of glycine and/or serine residues. In some embodiments, the linker comprises at least one glycine-rich region. For example, the glycine-rich region may comprise a sequence comprising [GS]n, wherein n is an integer between 1 and 10.

In some embodiments, the linker comprises polyethylene glycol. PEG chains of any length may be used as a linker, based on the degree of freedom desired for the binding agent. PEG of average molecular weight in the range of 100 to 10,000 Daltons (e.g., about 100, about 500, about 600, about 750, about 1,000, about 2,000, about 3,0000, about 4,000, about 5,000, about 6,000, about 7,000, about 8,000, about 9,000, and about 10,000 Daltons) are suitable for use herein.

In some embodiments, the linker further comprises a crosslinking site. In the case of polypeptide-based linkers, the crosslinking site may comprise amino acids which have side chains useful for crosslinking. For example, amino/amine groups (e.g., as found in lysine), thiol/sulfhydryl groups (e.g., as found in cysteine), and carboxylic acid groups (e.g., as found in aspartate, glutamate) are reactive side chains which can be exploited in a variety of crosslinking methods. In select embodiments, the crosslinking site may comprise a series of lysine residues. In select embodiments, the crosslinking site may comprise amino groups. In select embodiments, the crosslinking site may comprise one or more lysine residues and/or amino groups.

The grid may comprise any material used in EM sample grids known in the art (e.g., carbon, amorphous carbon, gold, or another material-on a metal (e.g., copper, gold, molybdenum, nickel)) to act as a substrate for the first layer. For example, the grid may comprise amorphous carbon, amorphous carbon coated copper, amorphous carbon coated gold, gold, or silicon nitride. The grid may be any size or shape, including for example square, rectangle, or circle. In some embodiments, the grid comprises a holey patterned substrate (e.g., a flat structure comprising holes or a mesh). The holes may be any size (e.g., width) or shape and may have irregular or regular spacing or arrangement.

The present disclosure also provides methods for manufacturing the above described grids. The methods comprise transferring a graphene sheet in aqueous solution to the surface of a grid substrate, wherein the grid substrate comprises holey patterned amorphous carbon, amorphous carbon coated copper, amorphous carbon coated gold, gold, or silicon nitride. In the methods of the invention the grid substrate(s) is immersed in the aqueous solution and the graphene sheet is floated in the aqueous solution. After matching and contacting the graphene sheet with the grid substrate(s) the grids are removed from the aqueous solution.

The methods may further comprise removing the remaining aqueous solution and drying the graphene coated grid. Suitable drying methods include treatment with heat, with or without vacuum treatment, or a combination thereof. The drying method may be completed for any period of time or repeated as necessary to completely dry the grids and remove any remaining aqueous solution.

In some embodiments, the graphene sheet comprises poly (methyl methacrylate) (PMMA) polymer. In such embodiments, the methods may further comprise removal of the PMMA. Removal of the PMMA may comprise washing with a solvent (e.g., acetone), heating without vacuum, heating under vacuum, or a combination thereof. The methods used for removal of the PMMA may be completed for any period of time or repeated as necessary to remove the majority of the PMMA from the surface of the grids.

The methods may further comprise oxidizing the graphene coated grid. The oxidation of the graphene coated grid may utilize any method which results in generating functional groups by which the binding agent can be tethered to the grid. For example, in some embodiments, the oxidation comprises the introduction of carboxylic acid functional groups useful for reacting with crosslinking agents and amino groups of the binding agent.

As such, the methods may further comprise crosslinking the binding agent to the oxidized graphene grid. The crosslinking may employ any of a variety of known methods and reagents (e.g., homobifunctional, beterobifunctional, photoreactive crosslinking reagents and appropriate buffers (e.g., MES, PBS)) depending on the functional group targets (e.g., amino, thiol, etc.). In some embodiments, the crosslinking reagents may include N-hydroxysuccinimide (NHS), N-hydroxysulfosuccinimide (sulfoNHS), 1-ethyl-3-(3-dimethylaminopropyl) carbodiimide (EDC), and mixtures thereof. In some embodiments, the crosslinking comprises N-ethyl-N′-(3-(dimethylamino) propyl) carbodiimide/N-hydroxysuccinimide (EDC/NHS)-mediated coupling. The crosslinking may be completed a single step or a multi-step process. In some embodiments, the crosslinking is completed in a single step.

The methods may further comprise incubating the grid with a sample comprising the target component (e.g., target protein), removing the excess sample, and visualizing the target component (e.g., target protein) on the grid by electron microscopy. In some embodiments, the methods further comprise vitrifying the target protein deposited on the grid. A variety of known ultrarapid freezing procedures are suitable for use in the disclosed methods, including, but not limited to, plunge freezing and high-pressure freezing.

In some embodiments, the target component is a target protein or multi-protein complex. The target protein multi-protein complex may comprise an affinity tag configured to interact with the binding agent. The affinity tag comprises any moiety (e.g., peptide, polynucleotide, or carbohydrate) which has affinity for and binds to the binding agent.

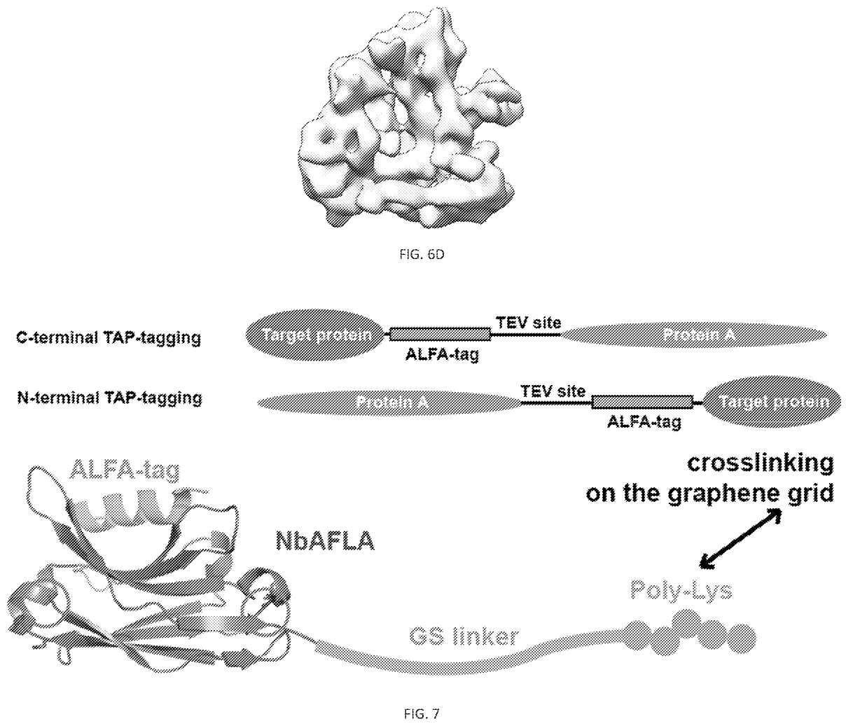

A wide variety of affinity tags and their cognate binding agents are known in the art and are suitable for use with the disclosed grids and methods. In some embodiments, the affinity tag is a protein domain tag (e.g., GST, MBP, SUMO, CBP, Halo, Nus A, FATT, ALFA). In some embodiments, the affinity tag is an epitope tag (e.g., FLAG, HA, V5, Myc, Strep, His, protein A). In some embodiments, the affinity tag comprises a CBP-tag or an ALFA-tag. In some embodiments, the tag comprises a TAP-tag, as described in FIG. 7.

The target protein or multi-protein complex may be obtained from a biological sample. In some embodiments, the target protein or multi-protein complex is obtained from a cell lysate.

### Methods

The present disclosure provides methods for preparing a target component (e.g., target protein) for structure analysis. The method may comprise incubating the grid disclosed herein with a sample comprising the target component, removing the excess sample, and visualizing the target component on the grid by electron microscopy (e.g., cryo-EM).

In some embodiments, the methods further comprise vitrifying the target component deposited on the grid. A variety of known ultrarapid freezing procedures are suitable for use in the disclosed methods, including, but not limited to, plunge freezing and high-pressure freezing.

The sample may be a biological sample. In some embodiments, the sample is a cell lysate. In some embodiments, the sample is obtained from a subject. The sample can be obtained from the subject using routine techniques known to those skilled in the art.

The sample may be used directly or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, partial purification of contaminants or non-target components, and the like.

In some embodiments, the target component is a target protein or multi-protein complex. The target protein or multi-protein complex may comprise an affinity tag configured to interact with the binding agent. The affinity tag comprises any moiety (e.g., peptide, polynucleotide, or carbohydrate) which has affinity for and binds to the binding agent.

A wide variety of affinity tags and their cognate binding agents are known in the art and are suitable for use with the disclosed grids and methods. In some embodiments, the affinity tag is a protein domain tag (e.g., GST, MBP, SUMO, CBP, Halo, Nus A, FATT, ALFA). In some embodiments, the affinity tag is an epitope tag (e.g., FLAG, HA, V5, Myc, Strep, His, protein A). In some embodiments, the affinity tag comprises a CBP-tag or an ALFA-tag. In some embodiments, the tag comprises a TAP-tag, as described in FIG. 7.

### Kits And Systems

The present disclosure provides kits for manufacturing the disclosed grids. The kits may include two or more of: a grid substrate; a graphene sheet; and a binding agent. In some embodiments, the kits contain the components necessary to manufacture a plurality of the disclosed grids in a single method or a plurality of grids over a plurality of methods.

The kits may further contain any necessary components to manufacture the grids, including, but not limited to, buffers, crosslinking agents, containers, and tools for use in incubating, supports for the grids, and the like. In some embodiments, the kits may contain the necessary components to produce and purify the binding agent. For example, the kits may contain polynucleotides encoding the binding agent, linker, and/or crosslinking sites, cells, transfection reagents, and the like.

Individual member components of the systems or kits may be physically packaged together or separately. The components of the systems or kits may be provided in bulk packages (e.g., multi-use packages) or single-use packages. The systems or kits provided herein are in suitable packaging. Suitable packaging includes, but is not limited to, vials, bottles, jars, flexible packaging, and the like.

In some embodiments, the kit further comprises instructions for using the components of the kit. The instructions are relevant materials or methodologies pertaining to the kit. The materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

Also within the scope of the present disclosure are systems that include a grid as disclosed herein or a kit as described above for making the disclosed grids and a sample. The systems may further contain any necessary components to use the grids for visualizing a target component from the sample, including, for example, reagents and materials necessary for vitrification, imaging software, and the like.

In some embodiments, the system further comprises instructions for using the components of the system. The instructions are relevant materials or methodologies pertaining to the system. The materials may include any combination of the following: background information, list of components and their availability information (purchase information, etc.), brief or detailed protocols for using the kit, trouble-shooting, references, technical support, and any other related documents. Instructions can be supplied with the system or as a separate member component, either as a paper form or an electronic form which may be supplied on computer readable memory device or downloaded from an internet website, or as recorded presentation.

It is understood that the disclosed systems or kits can be employed in connection with the disclosed methods. The system or kit may further contain additional containers or devices for use with the methods disclosed herein.

### EXAMPLES

The following are examples of the present invention and are not to be construed as limiting.

### Methods

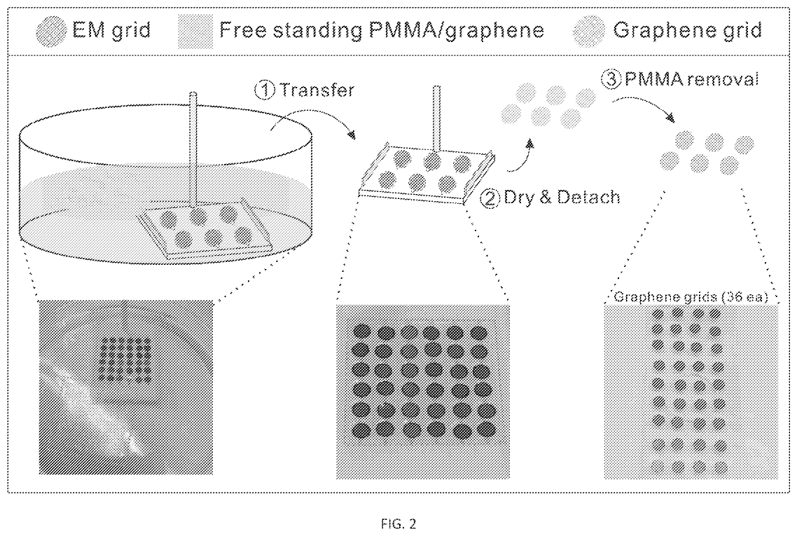

Making graphene-coated grids A total 36 Quantifoil holey carbon grids (e.g., Quantifoil grid) were placed on the 3D-printed grid transfer tool immersed in the DI water (FIG. 2). A free-standing PMMA/graphene was released and floated on the DI water. The PMMA/graphene layer was then carefully matched/transferred on the top of 36 grids using the tweezers/grid transfer tool and then scooped out of the water. The remaining water was drained with a cleaning wipe and placed in the oven (100° C., 30 min) to completely dry out the water. This step also increases contact between the graphene layer and grids. Fully dried PMMA/graphene coated grids were individually detached from the grid transfer tool and were immersed in the acetone solvent (50° C., 30 min, repeated 3 times) to dissolve and eliminate the PMMA layer. PMMA-removed graphene grids were transferred to the cover slide and baked in the oven (200° C., 12-24 hrs) to evaporate the remaining solvent and swelled polymers on the graphene.

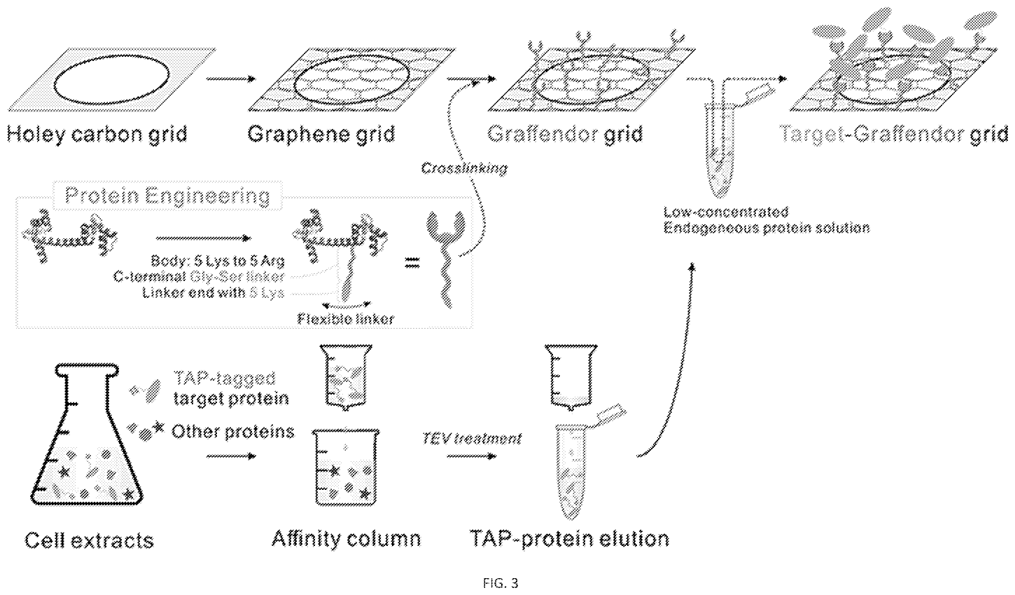

Protein engineering of Calmodulin Human calmodulin was genetically modified in three aspects (FIG. 3). The five surface exposed lysine residues were replaced with arginine residues to prevent the crosslinking between surface exposed lysine residues and EDC/NHS activated graphene grid. A 32 Gly-Ser (GS) linker was incorporated at the C-terminus of calmodulin to maintain the distance (˜100 Å) from the graphene surface, thereby providing flexibility to prevent the preferred orientation of particles. Five lysine residues were added at the C-terminus to serve as crosslinking sites.

Graffendor grids The as-prepared graphene grid was plasma-jet treated (or glow discharged treated) for 2 min to induce mild oxidation and generate functional groups (carboxylic acid group) for the cross-linking (FIG. 3). The plasma jet treated graphene grid was immersed in the 50 μL of EDC/Sulfo-NHS containing buffer solution (10 mL of 50 mM MES buffer solution (pH 6.5), mixed with EDC (9 mg) and Sulfo-NHS (26 mg)) for 15 min using the 3D printed reaction chamber. The semi-stable Sulfo-NHS crosslinked graphene grid was rinsed in a 50 mM MES buffer solution (pH 6.5) for 5 min to remove the excess EDC/NHS compound on the graphene grid surface. The Sulfo-NHS activated graphene grid was immersed in 50 AL of calmodulin containing buffer solution (0.5 mg/mL modified calmodulin in calmodulin binding buffer [30 mM HEPES (pH 7.0), 150 mM NaCl, 0.1% NP-40, 2 mM CaCl₂, 1 mM TCEP]) for 2 hrs. Alternatively, the as-prepared graphene grid was plasma-jet treated (or glow discharged treated) for 1 min to induce mild oxidation and generate functional groups (carboxylic acid group) for the cross-linking. The plasma jet treated graphene grid was immersed in 500 μL of fresh 5 mM EDC in 25 mM MES buffer solution (pH 6.5) for 5 min after which 500 μL of fresh 25 mM Sulfo-NHS in 25 mM MES buffer solution (pH 6.5) was added for 15 min using the 24-well glass plate. The semi-stable Sulfo-NHS crosslinked graphene grid was rinsed in a 25 mM MES buffer solution (pH 6.5) for 5 min to remove the excess EDC/NHS compound on the graphene grid surface. The Sulfo-NHS activated graphene grid was immersed in 500 μL of calmodulin containing buffer solution (0.5 mg/mL modified calmodulin in calmodulin binding buffer [30 mM HEPES (pH 7.0), 150 mM NaCl, 0.1% NP-40, 2 mM CaCl₂), 1 mM TCEP]) for 2 hrs. Using either methods, the prepared grids were stored at 4° C.

Loading grids Several liters of yeast cells containing a genetically engineered (e.g., TAP-tag at the C-terminus) target protein were lysed and applied to protein A chromatography. The eluate from the protein A chromatography was incubated with the grids for 1 hr (4° C., rotator) and washed. Following vitrification, the grids were imaged on a cryo-EM instrument.

### Example 1

### Preparing Chemically-Modified Graphene-Based Cryo-EM Grids

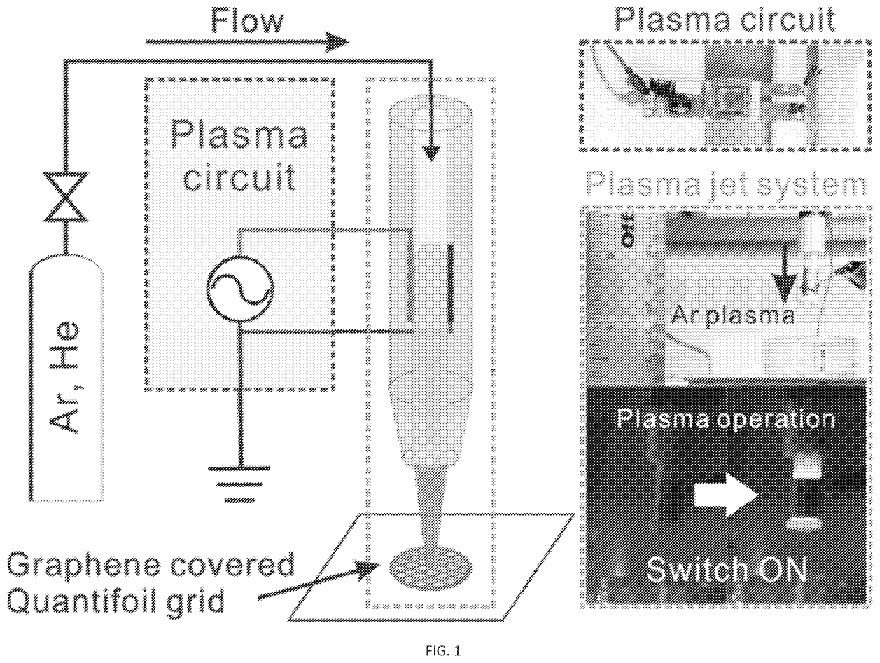

A graphene monolayer is hydrophobic, which requires the additional step to make its surface hydrophilic to prevent the curvature of water-droplet. As shown in FIG. 1, a plasma jet system was built to chemically modify the properties of graphene, including hydrophilicity, chemical activity, and functionality at the ambient environment. The conventional glow-discharger system requires several limitations to be widely used, such as a high-cost instrument setup, high vacuum condition (c.a. 0.26 mbar), and limited functionalization on the graphene surface. The disclosed plasma jet system overcomes these limitations with cost-effective device setup at the ambient condition, along with chemical modifications of the graphene surface (positive/negative charged or attaching functional groups) via a simple modification step (FIG. 1).

### Example 2

### Graphene Grids

An exemplary graphene transfer approach used herein is based on the polymer film assisted transfer method instead of polymer free graphene transferring method. There are several strengths of the polymer free transfer method, such that it can produce hyper clean graphene surface and high order crystallinity of the graphene, yet it requires installation of expensive instruments, which is not suitable for the individual laboratory. Polymer-assisted graphene transfer methods have the following advantages: 1) intuitive and easy-to-use method to generate graphene coated Quantifoil grids (e.g., graphene grids) at the individual laboratory level and 2) production of high-quality graphene grids with good surface coverage, cleanness, and mass production. The method takes 1.5 days to generate 36 graphene grids at once and utilizes a petri dish, a vacuum oven with a vacuum pump, a 1-inch×1-inch PMMA/graphene film, acetone, and a 3D-printed graphene transfer tool (FIG. 2). The detailed protocol is described in the Methods section. The baking step assists in producing clean graphene grids.

Both copper (Cu) Quantifoil grid and gold (Au) Quantifoil grids were tested for suitability for the graphene coating procedure. Gold Quantifoil grids are preferred for the graphene coating since copper Quantifoil grids due to oxidation concerns during 200° C. baking which resulted in color change and graphene damage.

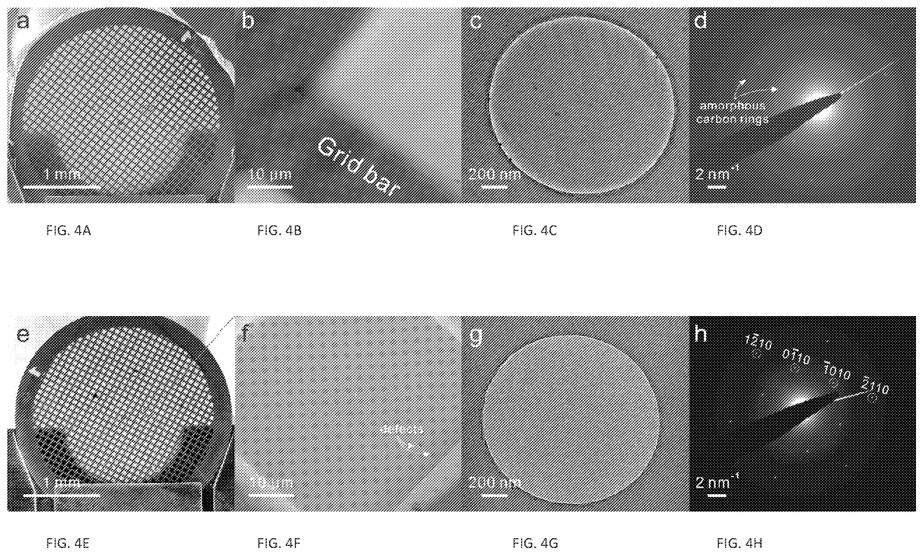

Comparison of PMMA/graphene versus PMMA-free graphene grids The graphene-coated Quantifoil grids were first characterized by using the Scanned electron microscopy (SEM) and bright-field transmission electron microscopy (BF TEM) to examine the coverage and surface quality of the graphene grids. In FIGS. 4A and 4B, the surface of the PMMA/graphene grid was fully covered with the thick PMMA layer (approximately 500 nm according to the product information), which even blocks the observation of holey patterns within the commercial Quantifoil grid (Au 200 mesh, 1.2/1.3). The TEM observation and following SAED pattern of the PMMA/graphene grid from the SEM analysis (FIGS. 4C-4D) showed a strong scattering pattern of the amorphous carbon that is originated from the covered PMMA layer. After the PMMA removal by rinsing/baking steps, the thick PMMA layer was mostly eliminated from the surface confirmed by SAM and BF TEM (FIGS. 4E-4H). The SEM image indicates that the graphene covers the holey-patterned carbon of the Quantifoil grid. The SAED diffraction image further demonstrates that the diffraction pattern of the amorphous carbon ring is minimized except some residual signals originated from the supporting holey carbon of the Quantifoil grid.

PMMA-assisted graphene transfer method is simple, highly reproducible, and resulted high coverage (˜95%) compared to graphene oxide deposition and coating method (less than 50%) on electron microscope (EM) grids. Graphene oxide deposition on EM grid utilizes an expensive tool (e.g., Langmuir Blodgett trough) and has multiple problems in reproducibility and consistency, such as multi-layer graphene oxide sheets and lack of coverage on the grid.

Characterization and quality evaluation of the graphene grid Former studies in generating graphene-coated grids and graphene oxide-coated grids have validated their coating quality of the grids by testing and visualizing the reference molecules, such as 20S proteasome, beta-galactosidase, and apo-ferritin, using cryo-EM. This approach may indirectly validate the quality of the produced graphene or graphene-oxide grids, but requires collection and examination of the cryo-EM data determine the quality of the grids. Therefore, quality validation tools useful before application of the specimen is highly demanded.

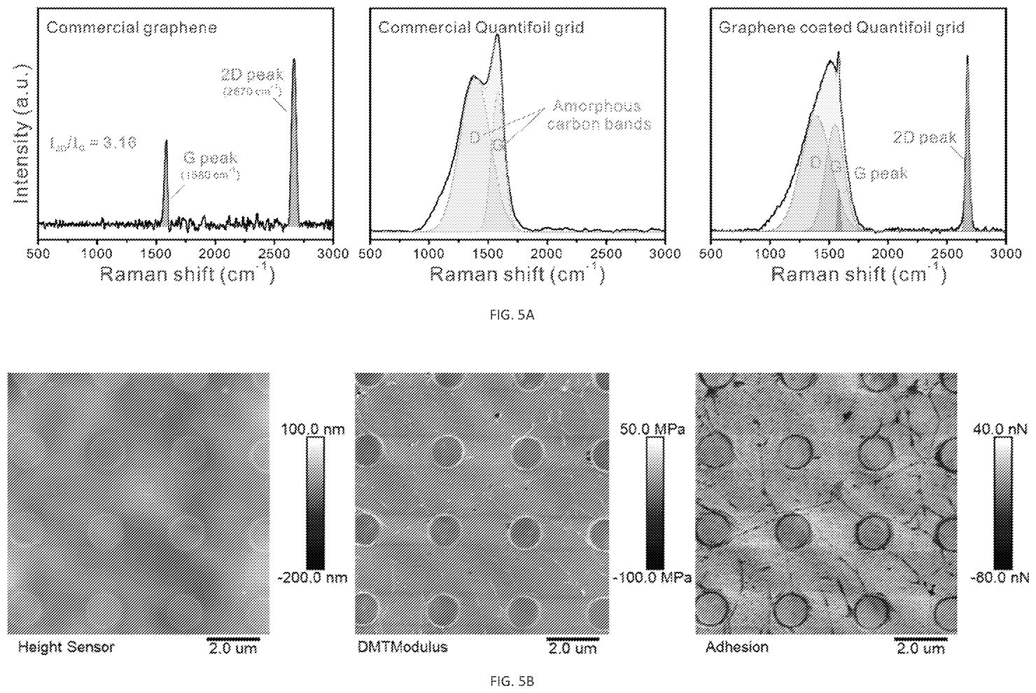

As quality control tools for the graphene-coated grids, Raman spectroscopy and atomic force microscopy (AFM) in addition to SEM and BF TEM were employed. The Raman spectra of the commercial graphene mono-layer (FIG. 5A; left), commercial Quantifoil grid (FIG. 5A; center), and the graphene-coated grid (FIG. 5A; right) were measured and compared. Non-defect and single-layered graphene of the commercial graphene grid was observed and validated with the absence of the D peak (1350 cm⁻¹; defect-induced second order Raman scattering) and the high peak ratio between the G peak (1580 cm⁻¹; explanation) and 2D peak (2670 cm⁻¹; explanation). It is generally known that the Raman intensity ratio of 2D/G (I_{2D}/I_{G}) over 2.0 denotes the single-layered characteristic of graphene. The Raman spectra of the Quantifoil grid (before the graphene transfer) showed large and broad D and G bands, which are derived from the holey patterned amorphous carbon. After graphene transfer on the Quantifoil grid, both characteristic peaks of the graphene layer (G, 2D) and band patterns of the Quantifoil grid (D, G) are shown up at the Raman spectra of the graphene-coated grid, which implies that the graphene monolayer is successfully transferred and coated on the Quantifoil grid. Therefore, the Raman spectroscopy and AFM, in addition to SEM and TEM images, were valuable validation tools to examine the quality of the graphene-coated grid after graphene transfer and before specimen application.

### Example 3

APC/C Complex Isolation and Imaging with Graffendor Grid

Before calmodulin was attached on the graphene-coated cryo-EM grid, it was genetically modified by replacing five lysine residues on calmodulin surface to arginine residues to prevent the unwanted chemical crosslinking between calmodulin and graphene surface (FIG. 3). A 32 Gly-Ser (GS) linker was incorporated at the C-terminus of calmodulin to maintain the distance (˜100 Å) from the graphene surface and to prevent the preferred orientation. Five lysine residues were added after the GS linker, which act as a major crosslinking site on the graphene surface. As a result, this modified calmodulin, when covalently attached on the graphene-coated cryo-EM grid, bound the CBP-containing proteins isolated directly from cells (FIG. 3).

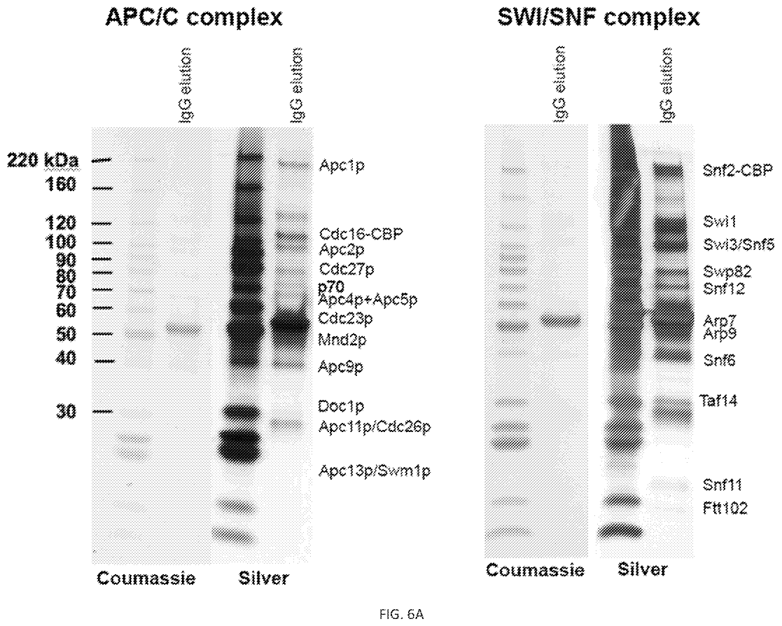

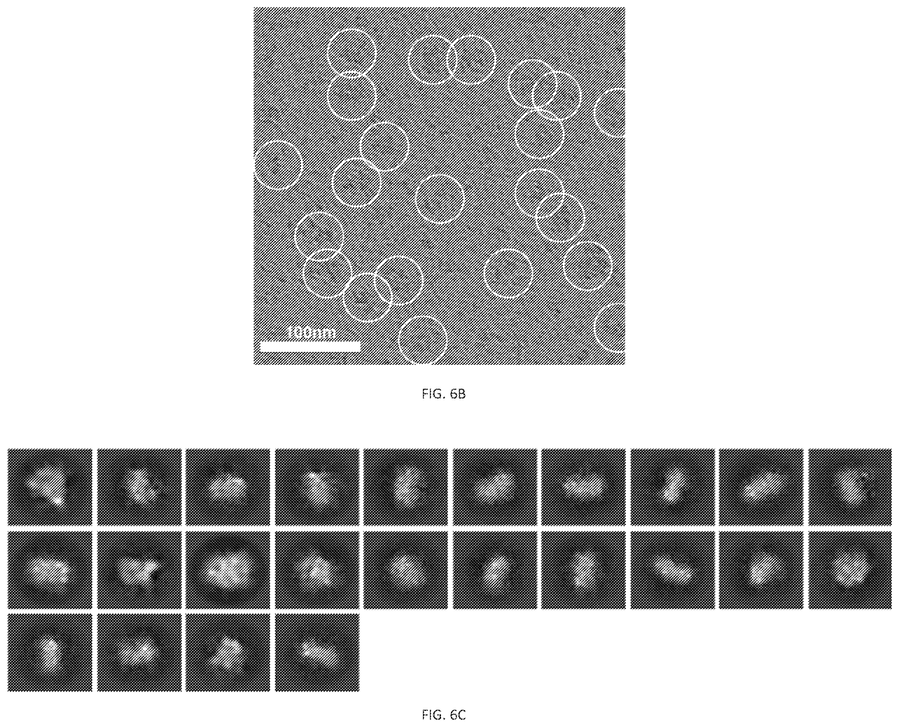

To validate the approach, the APC/C complex used as exemplary specimens. A 3 L yeast cell culture of Cdc16-TAP (APC/C) was harvested and applied into the IgG affinity chromatography. Incubating with TEV proteases released the target protein complexes into the IgG elution fraction. SDS-PAGE and subsequent Coomassie-staining resulted in the individual component of each complex being barely detectable, as shown in FIG. 6A. Additional silver-staining enhanced the contrast and visualized all components of the complexes. Using an exemplary Graffendor grid, the grid was incubated with the ten-fold diluted IgG eluate of the APC/C complex and vitrified after washing. The microscopic images of the Graffendor grid with the APC/C complex were screened and collected (525 images) using 200 KeV Glacios with the K2 direct detector. The micrographic image clearly visualized particles of the APC/C complex (FIG. 6B). A total of 8780 particles were selected via the TOPAZ program and performed subsequent 2D classification equipped in the cryoSPARC program suite (FIG. 6C). The ab-initio 3D reconstruction generated the low-resolution structure of the APC/C complex, which looks similar to the known structure (FIG. 6D).

### Example 4

### Graffendor-ALFA-Tag (GFD-A)

Mammalian cells express calmodulin as a calcium sensor and expressing target proteins with the CBP-tag may also recruit the endogenous calmodulin during the TAP-tag purification. As an alternate, an ALFA-nanobody based Graffendor grid comprising the pair of the ALFA-tag and ALFA-nanobody (NbALFA), which has a sub-nanomolar affinity (K_{D}=0.026 nM), for mammalian protein isolation, was designed. The ALFA-tag is 15 residues, and the NbALFA is a 13.5 kDa nanobody. The crystal structure of the ALFA-tag and NbALFA complex indicated that NbALFA is suitable for making the Graffendor grid by adding the GS linker and poly-lysine at the C-terminal end and mutating two lysine residues away from the ALFA-tag binding region (red circles) to arginine (FIG. 7).

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A grid for cryo-electron microscopy (EM) comprising a single first layer comprising graphene and a second layer comprising a binding agent configured to interact with a target protein, wherein the graphene covers greater than 90% of the grid.

2. The grid of claim 1, wherein the graphene covers greater than 95% of the grid with the single first layer.

3. The grid of claim 1 or claim 2, wherein the binding agent comprises a polypeptide.

4. The grid of any of claims 1-3, wherein the binding agent comprises calmodulin.

5. The grid of any of claims 1-3, wherein the binding agent comprises an antibody, a nanobody, or a fragment, derivative, or analog thereof.

6. The grid of any of claims 1-5, wherein the binding agent is attached to the first layer by a linker.

7. The grid of claim 6, wherein the linker is a flexible linker.

8. The grid of claim 6 or claim 7, wherein the linker comprises a glycine-serine rich polypeptide.

9. The grid of claim 6 or claim 7, wherein the linker comprises polyethylene glycol.

10. The grid of any of claims 6-9, wherein the linker further comprises a crosslinking site.

11. The grid of claim 10, wherein the crosslinking site comprises more than one lysine residue or amino group.

12. The grid of any of claims 1-11, wherein the target protein comprises an affinity tag configured to interact with the binding agent.

13. The grid of claim 12, wherein the affinity tag comprises a CBP-tag or an ALFA-tag.

14. The grid of any of claims 1-13, wherein the target protein is obtained from a cell lysate.

15. The grid of any of claims 1-14, wherein the grid comprises holey patterned amorphous carbon, amorphous carbon coated copper, amorphous carbon coated gold, gold, or silicon nitride as substrate for the first layer.

16. A method of preparing a target protein for structure analysis comprising:
incubating the grid of any of claims 1-15 with a sample comprising the target protein;
removing excess sample; and
visualizing the target protein on the grid by electron microscopy.

17. The method of claim 16, further comprising vitrifying the target protein deposited on the grid.

18. The method of claim 16 or claim 17, wherein the sample is a biological sample.

19. The method of any of claims 16-18, wherein the sample is a cell lysate.

20. The method of any of claims 16-19, wherein the sample is from a subject.

21. The method of any of claims 16-20, wherein the sample was partially purified prior to the incubation.

22. The method of any of claims 16-21, wherein the target protein comprises an affinity tag.

23. The method of claim 22, wherein the affinity tag comprises a CBP-tag or an ALFA-tag.

24. A method of manufacturing the grid of any of claims 1-15,
transferring a graphene sheet in aqueous solution to the surface of a grid substrate to form a graphene coated grid, wherein the grid substrate comprises holey patterned amorphous carbon, amorphous carbon coated copper, amorphous carbon coated gold, gold, or silicon nitride;
removing the aqueous solution;
drying the graphene coated grid;
oxidizing the graphene coated grid; and
crosslinking the binding agent to the oxidized graphene grid.

25. The method of claim 24, wherein the graphene sheet comprises poly (methyl methacrylate) (PMMA) polymer and the method further comprises removal of the PMMA.

26. The method of claim 25, wherein removal of the PMMA comprises washing with a solvent, heating with or without vacuum, or a combination thereof.

27. The method of any of claims 24-26, wherein the binding agent comprises a polypeptide.

28. The method of any of claims 24-27, wherein the binding agent comprises calmodulin.

29. The method of any of claims 24-27, wherein the binding agent comprises an antibody, a nanobody, or a fragment, derivative, and analog thereof.

30. The method of any of claims 24-29, wherein the binding agent comprises a linker.

31. The method of claim 30, wherein the linker is a flexible linker.

32. The method of claim 30 or claim 31, wherein the linker comprises a glycine-serine rich polypeptide.

33. The method of claim 30 or claim 31, wherein the linker comprises polyethylene glycol.

34. The method of any of claims 30-33, wherein the linker further comprises a crosslinking site.

35. The method of claim 34, wherein the crosslinking site comprises more than one lysine residue or amino group.

36. The method of any of claims 24-35, further comprising:
incubating the grid of any of claims 1-15 with a sample comprising the target protein;
removing excess sample; and
visualizing the target protein on the grid by electron microscopy.

37. The method of claim 36, further comprising vitrifying the target protein deposited on the grid.

38. The method of claim 36 or claim 37, wherein the sample is a biological sample.

39. The method of any of claims 36-38, wherein the sample is a cell lysate.

40. The method of any of claims 36-39, wherein the sample was partially purified prior to the incubation.

41. The method of any of claims 36-40, wherein the target protein comprises an affinity tag.

42. The method of claim 41, wherein the affinity tag comprises a CBP-tag or an ALFA-tag.

43. A kit comprising two or more of: a grid substrate; a graphene sheet; and a binding agent.

44. The kit of claim 43, wherein the grid substrate comprises holey patterned amorphous carbon, amorphous carbon coated copper, amorphous carbon coated gold, gold, or silicon nitride.

45. The kit of claim 43 or 44, wherein the binding agent comprises a polypeptide.

46. The kit of any of claims 43-45, wherein the binding agent comprises calmodulin.

47. The kit of any of claims 43-45, wherein the binding agent comprises an antibody, a nanobody, or a fragment, derivative, and analog thereof.

48. The kit of any of claims 43-47, wherein the binding agent comprises a linker.

49. The kit of claim 48, wherein the linker comprises a glycine-serine rich polypeptide.

50. The kit of claim 48, wherein the linker comprises polyethylene glycol.

51. The kit of any of claims 48-50, wherein the linker further comprises a crosslinking site.

52. The kit of claim 50, wherein the crosslinking site comprises more than one lysine residues or amino groups.

53. A system comprising:
a grid of any of claims 1-15 or a kit of any of claims 43-52; and
a sample.
